(19) United States
(12) Patent Application Publication
(10) Pub. No.: US 2005/0008590 A1
(43) Pub. Date: Jan. 13, 2005

(54) Effective method for regulating the appearance of skin

(75) Inventor: Ratan Chaudhuri, Lincoln Park, NJ (US)

Correspondence Address: MILLEN, WHITE, ZELANO & BRANIGAN, P.C., 2200 CLARENDON BLVD., SUITE 1400, ARLINGTON, VA, 22201, US

(73) Assignee: EM Industries, Hawthorne, NY (US)

(21) Appl. No.: 10/616,299

(22) Filed: Jul. 10, 2003

Related U.S. Application Data
(60) Provisional application No. 60/395,612, filed on Jul. 15, 2002.

(51) Int. Cl. A61K 7/00; A61K 31/7024

(52) U.S. Cl. 424/62; 514/25

(57) ABSTRACT

A method for regulating the appearance of skin comprising topically apply to said skin a composition comprising a cosmetically or pharmaceutically acceptable carrier and about 0.1% to about 40% of an extract comprising low molecular weight hydrolysable tannins.

### CROSS-REFERENCE TO RELATED APPLICATION

This application claims the benefit of Provisional Application Ser. No. 60/395,612 filed Jul. 15, 2002 and is related to application Ser. No. 10/120,156 entitled “Skin Lightening” and Provisional Application Ser. No. 60/349,224.

### FIELD OF THE INVENTION

This invention relates to novel methods for regulating the appearance of the skin by using matrix metalloprotease inhibitors.

### BACKGROUND OF THE INVENTION

Tissues, such as the skin, are composed of cells as well as an extensive network of macromolecules (e.g., polysaccharides and proteins) that make up the extracellular matrix (ECM) surrounding the cells. Specific cells within the ECM secrete two types of macromolecules-polysaccharide glycosaminoglycans (GAGs) and fibrous proteins (Alberts et al., Molecular Biology of the Cell, p. 802, New York: Garland Publishing, Inc., 1989). Polysaccharide GAGs are long, unbranched polysaccharides composed of repeating disaccharide units. These structures are sometimes linked to a core protein to form larger structures known as proteoglycans (Alberts et al., Molecular Biology of the Cell, p. 803, New York: Garland Publishing, Inc., 1989). The fibrous proteins of the ECM consist of structural proteins such as elastin and collagen, which accounts for about 70% of the dry weigh of the dermis (Lahmann et al., Lancet, 357(9260):935-6, 2001), and adhesive proteins such as fibronectin and laminin. The structure of the ECM is typically characterized by the GAGs and proteoglycans forming a gel-like substance in which the fibrous proteins are embedded (Alberts et al., Molecular Biology of the Cell, p. 803, New York: Garland Publishing, Inc., 1989).

As skin ages, its physiology goes through many changes. These changes are caused by structural and/or functional transformations of the ECM that usually take place during the passage of time and during over exposure to sunlight. Aging or long-term exposure to UV light is associated with i) a decrease in collagen synthesis; ii) an increase in matrix metalloprotease (MMP) activity; and iii) a reduction of the expression of tissue inhibitors of metalloproteases (TIMP). The combined actions of these factors result in a progressive degradation of the macromolecules and thus the structure of the ECM (Thibodeau, Allured's Cosmetic & Toiletries Magazine, 115(11):75-82, 2000).

The integrity of ECM components, in particular collagen, is important to ensure that the epidermal layer of the skin firmly anchors to the dermal layer of the skin. Degradation of collagen at the dermo-epidermal junction by proteases (e.g., matrix metalloproteases) weakens dermal adhesion, which eventually leads to the appearance of wrinkles and a reduction in the firmness and elasticity of the skin (Thibodeau, Allured's Cosmetic & Toiletries Magazine, 115(11):75-82, 2000).

Matrix metalloproteases (MMPs, e.g., gelatinase, Collagenase and stromelysin), which digest collagen, gelatin (denatured collagen) and other components of the ECM, are important for both normal development and carcinogenesis. When cells from one tissue invade a neighboring tissue, as in angiogenesis, wound healing, fetal tissue development and metastasis of tumors, MMPs are released to facilitate the breakdown of barriers opposing the invading cells.

There are a number of different MMPs that are specific for the various ECM components. For instance, gelatinase A (MMP-2) is primarily responsible for the degradation of the helical domains of type IV collagen, the principal collagen of basement membranes, while interstitial collagenase (MMP-1) is more selective for type I collagen (Thibodeau, Allured's Cosmetic & Toiletries Magazine, 115(11):75-82, 2000). MMPs are regulated by a number of inhibitor proteins, termed tissue inhibitors of metalloprotease (TIMPs), and by the relationship between the MMPs themselves. Further, TIMPs play a key role in regulating growth, invasion, and metastasis of neoplastic cells.

The enzymatic balance between MMPs is naturally controlled by the presence of tissue inhibitors of matrix metalloproteases. Aging and environmental insults, such as UV light, disrupt this equilibrium in favor of the over expression of these enzymes. MMPs of type I (i.e., interstitial collagenase, MMP-1) are responsible for degradation of skin collagen fibers, particularly during photo-damage, and this degradation of collagen fibers contributes to the visible effects of UV damage (i.e., wrinkling, loss of elasticity and dilation of surface micro-capillary vessels).

Therefore, in order to reduce the visible affects caused by UV exposure and MMPs, it is necessary to inhibit or reduce the activity of MMPs. This can be achieved by regulating gene transcription of the MMPs or by inactivating MMPs by use of inhibitors.

Thus, there is a need for an effective method to inhibit MMP activity, in particular UV-induced MMP activity, and thus regulate or enhance the appearance of the skin.

Compositions comprising collagenase inhibitors to treat the skin are known in the art. For example, U.S. Pat. No. 5,614,489 relates to skin treatment compositions containing a collagenase and/or elastase inhibitor and a method for reducing evidence of wrinkles and aging by apply such compositions to the skin. In addition, U.S. Pat. No. 6,365,630 relates to compositions and methods for ameliorating the effects of UVA and UVB radiation from the sun. The recited compositions comprise UVA and UVB blockers and MMP inhibitors. However, neither of these references provides for the use of compositions based on extracts of Phyllanthus emblica in methods to treat skin conditions associated with aging and exposure to UV radiation.

Furthermore, U.S. Pat. No. 6,362,167 relates to a method for blocking, in an animal, free radical processes by using an antioxidant composition comprising an extract of Phyllanthus emblica. Such free radical processes have been implicated in disorders such as premature aging, aging, and age related diseases. However, this patent does not suggest or provide for a method for treating skin conditions associated with UV-induced collagenase activity.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1 is a bar graph showing a significant reduction in collagenase expression in human skin fibroblasts in the presence of a standardized extract of Phyllanthus emblica (50 μg/ml) when compared to control skin fibroblasts (without the a standardized extract of Phyllanthus emblica).

FIG. 2 is a bar graph showing the dose- and time-dependent inhibition (40-75%) of gelatinase/collagenase activity in the presence of a standardized extract of Phyllanthus emblica (150-300 μg/ml).

### DETAILED DESCRIPTION OF THE INVENTION

It has been discovered that an extract, preferably a standardized extract of Phyllanthus emblica (syn. Emblica officinalis) has significant collagenase inhibitory activity, and thus can be used in effective methods for regulating or improving the appearance of human skin. A standardized extract of Phyllanthus emblica is described in U.S. patent application Ser. No. 10/120,156 (filed Apr. 12, 2002).

The preferred composition used in the present invention comprises an extract of low molecular weight hydrolyzable tannins (0.1 to 40% w/w). More specifically, the composition comprises Emblicanin A, Emblicanin B, Punigluconin, Pedunculagin and a cosmetically or pharmaceutically acceptable carrier (60 to 99.9% w/w). Commercially, the above-described tannin composition can be obtained from a suitable plant source such as Phyllanthus emblica (syn. Emblica officinalis) or other suitable sources.

In French patent 2730408 published Aug. 14, 1996, compositions are proposed based on extracts of fruits among which is Phyllantus emblica (syn.Emblica officinalis). The composition may be based on a dilute-alcoholic extract obtained from the Phyllantus emblica or an extract obtained, for example by merely pressing the fruit.

Both the extracts obtained by pressing and the extracts obtained by alcoholic maceration may then be concentrated at a moderate temperature under reduced pressure, preferably less than 50° C., then optionally brought to the dry state by freeze-drying or any other method under reduced pressure and at a temperature that is lower than 50° C. so as to avoid degrading the active ingredients of the fruit.

In this French patent, however, there is no indication of the composition of the extracts. Conversely, in U.S. Pat. No. 6,124,268, Ghosal, issued Sep. 26, 2000 entitled “Natural Oxidant Compositions, Method For Obtaining Same And Cosmetic, Pharmaceutical and Nutritional Formulations Thereof” there is set forth the chemical composition of extracts of Emblica officinalis obtained by extracting the fresh fruit at elevated temperatures, e.g. 70° C., using a very dilute aqueous or alcoholic-water salt solution, e.g. 0.1 to 5%. By this extraction process, in the presence of sodium chloride, for example, hydrolysis of the glycocidic enzymes in the plant is prevented and the product is protected from microbial infestation.

In the Ghosal patent, the blend of constituents are described under the name of “CAPROS”, with claim 8, for example, of the patent setting forth the composition as follows:

An blend consisting essentially of, by weight, (1) and (2) about 35-55% of the gallic/ellagic acid derivatives of 2-keto-glucono-δ-lactone; (3) about 4-15% of 2,3-di-O-galloyl-4,6-(S)-hexahydroxydiphenoylgluconic acid; (4) about 10-20% of 2,3,4,6-bis-(S)-hexahydroxydiphenoyl-D-glucose; (5) about 5-15% of 3′,4′,5,7-tetrahydroxyflavone-3-O-rhamnoglucoside; and (6) about 10-30% of tannoids of gallic/ellagic acid.

The common names of the enumerated compounds are (1) Emblicanin A, (2) Emblicanin B, (3) Purigluconin, (4) Pedunculagin and (5) Rutin.

The preferred composition used in the present invention comprises a modification of the CAPROS composition, comprising a standardized extract of low molecular weight (<1000) hydrolyzable tannins, over 40%, preferably 50-80% w/w of Emblicanin A, Emblicanin B, Pedunculagin, and Punigluconin with less than 1% flavonoids and especially no or low levels (<1%, w/w) of Rutin (3′,4′,5,7-tetrahydroxyflavone-3-O-rhamnoglucoside). A more detailed description of a composition except for the content of flavonoids and Rutin is discussed with pages 28-30 of the August 2001 issue of Soap, Perfumery and Cosmetics, the article having the title Ingredients/Emblica, Bearing Fruit, by Ratan K. Chaudhuri. The preferred concentrations of Rutin in the standardized extract are less than 1.0%, less than 0.01%, less than 0.001% and less than 0.0001%, with a value of 0.01 to 0.001% being particularly preferred. As for the other ingredients, the most preferred concentrations of the other components are on a percent by weight basis of the total dried extract:

| | Most Preferred Concentrations |
|---|---|
| | % by weight |
| Emblicanin A | 20-35 |
| Emblicanin B | 10-20 |
| Pedunculagin | 15-30 |
| Punigluconin | 3-12 |

The standardized composition preferably exhibits average percentage deviations from these preferred values of:

| | Preferred Deviation | Most Preferred Deviation |
|---|---|---|
| Emblicanin A | ±10% | ±5% |
| Emblicanin B | ±10% | ±5% |
| Pedunculagin | ±10% | ±5% |
| Punigluconin | ±10% | ±5% |

The composition can be obtained by removal of Rutin by reversed-phase column chromatography or HPLC using a solvent system of acetonitrile, water/phosphoric acid (20/80/1) or other solvent combinations as it elutes faster than the low molecular-weight tannins. Also, by selection of geographical location, the Phyllanthus emblica fruit extract may provide a substantially lower level of Rutin (<1.0%, w/w). It has been observed that medium-sized fruits collected from some parts of eastern India, during October-November, after water extraction and drying, yielded the preferred composition as a powder with the desired low content of Rutin. Accordingly, by analyzing the Rutin content of extracts and selecting such extracts that contain the desired low content of Rutin, it is possible to prepare a standardized extract.

The resultant standardized extract powdery material is then incorporated in a cosmetically or pharmaceutically acceptable carrier, preferably having a pH ranging from about 3 to 6.5. The carrier is any conventional carrier for topical administration and is preferably employed in a concentration of about 60%-99.9%, preferably 90% to 99.7%, and more preferably 95% to 99.5%. (In other words, the concentration of the composition of the present invention is generally about 0.1 to 40% by weight, preferably 0.3 to 10% by weight, and more preferably 0.5 to 5% by weight.).

The composition can be used to treat or regulate skin conditions characterized as visible and/or tactile discontinuities in the skin, signs of aging, and visible and/or tactile discontinuities in the skin associated with skin aging (e.g., fine lines, wrinkles, surface roughness, dryness and other texture discontinuities associated with aged skin).

In addition to or included with the above mentioned conditions, this composition can be of use for delaying the appearance of fine lines, enhancing extracellular matrix cohesion, reducing the appearance of spider veins, reducing skin redness, improving skin firmness and elasticity, and reducing damage caused by over exposure to the sun.

The composition used in the present invention can be optionally mixed with other suitable skin care agents, either known prior to the present disclosure as well as those which will be invented in the future. For example, the skin care agents, which can be used include but are not limited to conventional skin care excipients as well as additional photoprotective agents and skin lightening agents.

As for the additional photoprotective agents, if sunscreens are added, suitable sunscreens include any agent capable of protecting the skin from UV radiation including, for example, butyl methoxydibenzoylmethane, cinoxate, benzophenone-8, homosalate, menthyl anthranilate, octocrylene, ethyhexyl methoxycinnamate, ethylhexyl salicylate, benzophenone-3, ethylhexyl dimethyl PABA, glyceryl PABA, phenylbenzimidazole sulfonic acid, benzophenone-4, ethyhexyl triazone, diethylhexyl butamido triazone, bisimidazylate etc.

In addition to photoprotective agents, the compositions and formulations of the present invention are effective for skin whitening and can be optionally blended with other skin whitening agents. For example, the skin whitening products which can be combined include but are not limited to cysteine, 4-thioresorcin, 3-aminotyrosine, 5-hydroxy-2-hydroxymethyl-γ-pyridone, fomesjaponicus and ganoderma extracts, kojic acid, glabridin inhibited tyrosinase, glycyrrhizinic acid, hydroquinone-α-glucoside, catharanthus roseus extract, proteoglycans, proteinase inhibitors, oligopeptides, betaines, and methyl 4-benzyloxy-2-hydroxybenzoate and 4-benzyloxy-2-hydroxybenzoic acid.

For the purposes of providing a topical formulation of the composition of the present invention, any of the known topical excipients can be used therewith such as mineral oils, emulsifying agents, preservatives, anti-oxidants, skin penetrants, etc., including but not limited to the various topical excipients which are utilized in U.S. Pat. No. 6,124,268 and the references discussed above. The compositions can be employed as typical topical compositions utilized in the dermatological and cosmetic fields, e.g., lotions, gels, emulsions, sprays, sticky liposome coacervates, etc.

With respect to the amount of the topical composition which is applied to the skin, it should be a sufficient amount and for a sufficient period of time to visibly change or improve the appearance of the skin. On a molecular level, the amount of the topical composition which is applied to the skin should be a sufficient amount and for a sufficient period of time to inhibit or decrease the expression or function of matrix metalloproteases, such as Collagenase (MMP-1). Preferably the topical composition contains an amount of about 0.3 to about 5.0% by weight of the composition in a formulated product and preferably for at least about once per day for a period of preferably at least about two weeks.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosures of all applications, patents and publications, cited above or below is hereby incorporated by reference.

### EXAMPLE I

### Measurement of Collagenase (MMP-1) Expression in Human Skin Fibroblast Cells in the Presence of a Standardized Extract of Phyllanthus emblica

The object of this study was to quantify the collagenase expression in human skin fibroblast cells after incubation with different concentrations of a standardized extract of Phyllanthus emblica using the MMP-1 ELISA kit from Oncogene.

The MMP-1 ELISA is a “sandwich” enzyme immunoassay employing two monoclonal antibodies. Specifically, a monoclonal antibody specific for human MMP-1 protein is immobilized onto the surface of the wells of a microtiter plate provided in the kit. The sample to be assayed (test samples and standards) are pipetted into the wells and any human MMP-1 protein present in the sample binds to the captured antibody. Unbound material is washed away and a monoclonal, horseradish peroxidase (HRP)-conjugate anti-MMP-1 antibody is added to the wells. Following an incubation period and a wash step with buffer, a chromogenic substrate is added to the wells. The horseradish peroxidase catalyzes the conversion of the cromogenic substrate, tetra-methylbenzidine (TMB) from a colorless solution to a blue solution (the color changes to yellow after the addition of stopping reagent). The intensity of color is proportional to the amount of human MMP-1 protein in the test sample. The color reaction product is then quantified using a spectrophotometer.

Quantitation is determined by construction of a standard curve using known concentrations of human MMP-1 protein. By comparing the absorbance obtained from a sample containing an unknown amount of human MMP-1 protein with the absorbance obtained from the standards, the concentration of human MMP-1 protein in the test sample can be determined.

### Materials and Methods

Reagents

All of the reagents listed below were obtained form Oncogene (Cambridge, Mass. 02142, USA). The assay kit contains the following:
Coated Microtiter Plate: 96 removable wells coated with mouse anti-human MMP-1 monoclonal antibody. 1 lid to cover microplate during incubations.
ProMMP-1 Protein Standard: 3.6 ng of human proMMP-1 lyophilized with preservatives.
MMP-1 Conjugate: 0.6 ml of anti-MMP-1 monoclonal antibody conjugated to horseradish peroxidase (HRP). Dilute with 12 ml of assay buffer.
Assay Buffer: 2 bottles of sodium phosphate buffer containing BSA lyophilized. The contents of each bottle will be reconstituted in 20 ml of deionized, distilled water.
Wash Buffer Concentrate: 12 ml of a 25× concentrated solution of sodium phosphate buffer, pH 7.0.
Color Reagent: 12 ml of the chromagenic substrate, tetra methylbenzidine (TMB). This reagent is light sensitive and should be protected from direct sunlight or UV sources.
Stop Solution: 2.5 N sulfuric acid.

Cell Cultures

Human fibroblasts were obtained from biopsies of healthy skin from shaved forearms. The cells were grown in DMEM medium containing 10% fetal calf serum and supplemented with penicillin (100000 U/I) and streptomycin (100 mg/l). Fibroblasts were grown to confluence in 75 cm² culture dishes (Falcon® Plastics, Calif., USA) at 37 C. under a water-saturated sterile atmosphere containing 5% CO₂. All experiments were done on subcultures between the fifth and tenth transfer.

Assay Protocol

For the assay, the cultured cells above were trypsinized and placed into 24 well plates and incubated over night at 37° C. in 5% CO₂ before the assay. The cell density used was 3×10⁵ cells per well at the moment of experimentation. After addition of the standardized extract of Phyllanthus emblica (MMP inhibitor), cell cultures were incubated for 24 and 48 hours at 37° C. in 5% CO₂. The assay was performed as follows:
1. Prepare all samples, controls, standards and reagents as described in the previous sections.
2. Remove the appropriate number of microtiter wells from the pouch and place them into the empty well holder. Return any unused wells to the original pouch, refold, seal and store at 2-8° C. for up to 1 week.
3. Pipette 100 μl of standard or sample into each well.
4. Cover the plate with the lid provided, and incubate the plate at room temperature for 2 hours without shaking.
5. Aspirate each well and wash wells 5 times with 1× Wash Buffer making sure each well is filled completely. Each well is washed by filling with 400 μl of 1× wash buffer. It is essential to remove completely the wash buffer after each step and to ensure that the wells do not contain any buffer after the last wash. This can be achieved by inverting the plate and tapping it on paper towels.
6. Pipette 100 μl of the MMP-1 conjugate into each well. Cover the plate with the lid provided, and incubate the plate at room temperature for 1 hour without shaking.
7. Aspirate each well and wash the wells 5 times with 1× wash buffer making sure each well is filled completely. Follow the procedure outlined in step 5.
8. Pipette 100 μl of color reagent into each well. Cover the plate with the lid and incubate the plate in the dark at room temperature for 30 minutes without shaking.
9. Stop the reaction by adding 100 μl of stop solution (2.5 N sulfuric acid) into each well in the same order as the color reagent was added. If the color does not appear uniform in the well, gently tap the microtiter plate frame to ensure thorough mixing.
10. Measure absorbance in each well using a spectrophotometric plate reader at dual wavelengths of 450/595 nm (540 nm can be used as an alternative reference wavelength). If wavelength reference is not available, subtract the readings at 595 nm or 540 nm from the readings at 450 nm. This subtraction will correct for optical imperfections in the plate. Readings made directly at 450 nm without correction may be higher and less accurate. Wells must be read within 30 minutes of adding the stop solution.

Results

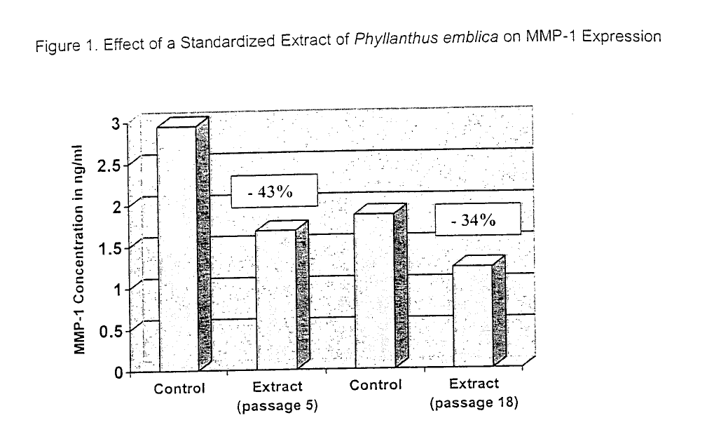

Results of this study are summarized in Table 1 and FIG. 1. Experiments were done using human skin fibroblast cell of fifth and eighteenth passages. Data show that the effect of the standardized extract of Phyllanthus emblica on matrix metalloprotease MMP-1 (collagenase) expression after 48 hours of incubation in two different experiments is comparable. Both experiments showed a significant decrease (about 39%) in MMP-1 expression.

TABLE 1
Effect of a standardized extract of Phyllanthus emblica on
MMP-1 Expression

| | Optical | Optical | | Standard | |
|---|---|---|---|---|---|
| | Density | Density | Average | Deviation | ½ SD |
| Fibroblasts | | | | | |
| (5^{th} passage) | | | | | |
| Control | 2.98 | 2.91 | 2.95 | 0.049 | 0.025 |
| Standardized extract | 1.68 | 1.68 | 1.68 | 0 | 0 |
| of Phyllanthus | | | | | |
| emblica (50 μg/ml) | | | | | |
| Fibroblasts | | | | | |
| (18^{th} passage) | | | | | |
| Control | 1.77 | 1.95 | 1.86 | 0.127 | 0.064 |
| Standardized extract | 1.17 | 1.27 | 1.22 | 0.071 | 0.035 |
| of Phyllanthus | | | | | |
| emblica (50 μg/ml) | | | | | |

MMP-1 (collagenase) expression is decreased by about 39% after incubation of human skin fibroblasts for 48 hours in the presence of a standardized extract of Phyllanthus emblica (50 μg/ml).

### EXAMPLE II

### Measurement of the Collagenase Inhibitory Activity of a Standardized Extract of Phyllanthus emblica

The object of this study was to quantify the collagenase activity in human skin fibroblast cells after incubation with different concentrations of a standardized extract of Phyllanthus emblica using a gelatinase/collagenase assay kit from EnzChek®.

Molecular Probes' EnzChek® gelatinase/collagenase assay kit (E-12055) contains DQ ggelatin, fluorescein conjugate (gelatin is heavily labeled with fluorscein that the fluorescence is quenched). The increase of fluorescence is proportional to proteolytic activity and can be monitored with a fluorescence microplate reader. Using 100 μg/ml DQ gelatin and a two-hour incubation period, the assay can detect the activity of the enzyme down to a final concentration of 2×10⁻³ U/ml (7 ng of protein/ml). One unit if defined as the amount of enzyme required to liberate 1 mmole of L-leucine equivalents from collagen in 5 hours at 37° C., pH 7.5. An incubation time of about 24 hours can increase the sensitivity to about 10-fold. At high enzyme concentrations (e.g., 0.2-0.4 U/ml), incubation times can be as short as about 15 minutes.

### Materials and Methods

The EnzChek gelatinase/collagenase assay kit provides the following components:
DQ Gelatin from pig skin, flourescein conjugate (Component A): five vials each containing 1 mg substrate lyophilized from 1 ml of phosphate buffered saline (PBS), pH 7.2.
10× Reaction Buffer (Component B): 50 ml of 0.5 M Tril-HCl,1.5 MNaCl₂, 2 mM sodium azide pH 7.6.
2,20-Phenanthroline, monohydrate (Component C): approximately 30 mg (MW=198.2), a general metalloproteinase inhibitor.
Collagenase, Type IV from Clostridium histolyticum (Component D): 500 U. One unit is defined as the amount of enzyme required to liberate 1 μmole of L-leucine equivalents from collagen in 5 hours at 37 C., pH 7.5.

Assay Protocol
1. Prepare a 1 mg/ml stock solution of DQ gelatin by adding 1 ml of deionized hater directly to one of the five vials containing the lyophilized substrate. It may be necessary to agitate the sample in an ultrasonic water bath for about 5 minutes and heat to 50° C. to facilitate dissolution. A 2.5-20 μl volume will be used for each 200 μl volume reaction; thus this stock solution provides substrate sufficient for approximately 50-400 assays using a fluorescence reader and assay volumes of 200 μl per microplate well. Proportionally fewer assays will be possible if the reaction is scaled up to accommodate fluorometer cuvettes. Reconstitued DQ gelatin may be stored in the dark at 4° C. with the addition of sodium azide to a final concentration of 2 mM.
2. Prepare 1× reaction buffer. Dilute 2 mL of the 10× reaction buffer in 18 mL deionized water. This 20 ml volume is enough working 1× reaction buffer for at least fifty 200 μl assays with about 10 ml excess for performing dilutions and preparing working solutions.
3. If using the Clostridium collagenase, prepare a 1000 U/ml stock solution by dissolving the contents of the vial (Component D) in 0.5 ml deionized water. Reconstituted Clostridium collagenase can be frozen in aliquots and stored at 20° C. for at least six months without significant loss of activity.
4. Weigh out 9.9 mg of 2,20-phenanthroling from the approximately 30 mg provided (Component C) and dissolve in 25 μl of ethanol or N,N-dimethylformamide. Prepare a 10 mM working solution by adding 10 μl of this solution to 2 ml of 1× reaction buffer prepared in step 2.
5. Dilute the inhibitor of interest in 1× reaction buffer. An 80 μl volume of inhibitor will be used for each 200 μl reaction. The provided inhibitor, 2,20-phenanthroline, can serve as a control inhibitor. Make dilutions of 2,20-phenanthroline from the 10 mM working solution from step 4. It was determined that 0.1 mM 2,20-phenanthroline is a suitable concentration for use with Clostridium collagenase at 0.2 U/ml; the appropriate concentration range may be different for the other enzymes. Include a no-inhibitor control for all enzymes being assayed.
6. Add 80 μl of the diluted inhibitor (or no inhibitor as control) to each assay well.
7. Next, add 20 μl of DQ gelatin stock solution to each assay well (see step 6).
8. Dilute the enzyme of interest in 1× reaction buffer. Add 100 μl of the diluted enzyme, or 100 μl of 1× reaction buffer as a blank, to the sample wells preloaded with substrate and inhibitor.
9. Incubate the samples at room temperature, protected from light, for approximately 1-2 hours. Because the reaction is continuous (not terminated), fluorescence may be measured at multiple time points.
10. Measure the fluorescence intensity in a fluorescence microplate reader equipped with standard fluorescein filters. Digested products from the DQ gelatin has absorption maxima at about 495 nm and fluorescence emission maxima at about 515 nm.
11. For each time point, correct for background fluorescence by subtracting the values derived from the no-enzyme control.

Results

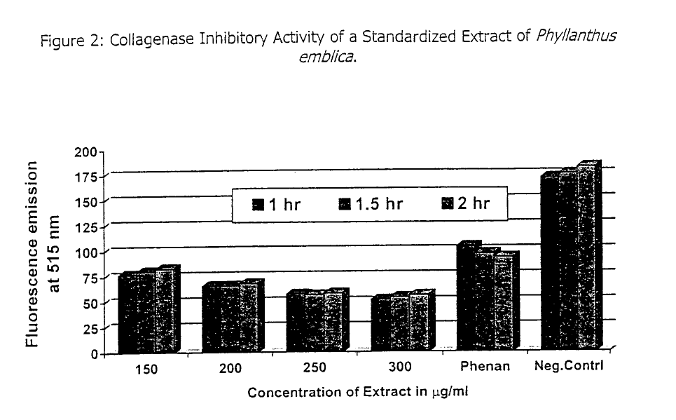

Quantification of gelatinase/collagenase inhibitory activity of a standardized extract of Phyllanthus emblica was determined by using EnzChek® gelatinase/collagenase kit (E-12055) from Molecular Probe by measuring the substrate fluorescence emission at 515 nm. 1,10-phenanthroline (Phenan) was used as a positive control and collagenase without inhibitor was used as a negative control. A dose- and time-dependent inhibition (55-70%) of gelatinase/collagenase activity was observed with a standardized extract of Phyllanthus emblica (150-300 μg/ml). Results of this study are summarized in FIG. 2.

Degradation of collagen fibers by UV induced MMP activity contributes to the visible effects of UV damage (i.e., wrinkling, loss of elasticity, and dilation of surface micro-capillary vessels). As shown in FIG. 2, the standardized extract of Phyllanthus emblica provides a dose- and time-dependent inhibition (55-70%) of gelatinase/collagenase activity. Therefore, it is expected that a standardized extract of Phyllanthus emblica will protect human skin against photo-induced damage.

The preceding examples can be repeated with similar success by substituting the generically or specifically described reactants and/or operating conditions of this invention for those used in the preceding examples.

The entire disclosure of all applications, patents and publications, cited above are hereby incorporated by reference.

From the foregoing description, one skilled in the art can easily ascertain the essential characteristics of this invention and, without departing from the spirit and scope thereof, can make various changes and modifications of the invention to adapt it to various usages and conditions.

1. A method for regulating the appearance of skin comprising topically apply to said skin a composition comprising a cosmetically or pharmaceutically acceptable carrier and about 0.1% to about 40% of an extract comprising low molecular weight hydrolysable tannins.

2. The method of claim 1, wherein said extract comprises over 40% of Emblicanin A, Emblicanin B, Pedunculagin, and Punigluconin.

3. The method of claim 2, wherein said extract comprises about 50% to about 80% of Emblicanin A, Emblicanin B, Pedunculagin, and Punigluconin.

4. The method of claim 2 or 3, wherein said extract comprises less than 1% Rutin.

5. A method for inhibiting or decreasing the expression or function of matrix metalloproteases in the skin comprising topically apply to said skin a composition comprising a cosmetically or pharmaceutically acceptable carrier and about 0.1% to about 40% of an extract comprising low molecular weight hydrolysable tannins.

6. The method of claim 5, wherein said extract comprises over 40% of Emblicanin A, Emblicanin B, Pedunculagin, and Punigluconin.

7. The method of claim 6, wherein said extract comprises about 50% to about 80% of Emblicanin A, Emblicanin B, Pedunculagin, and Punigluconin.

8. The method of claim 6 or 7, wherein said extract comprises less than 1% Rutin.
